(19) United States
(12) Patent Application Publication
(10) Pub. No.: US 2007/0088374 A1
(43) Pub. Date: Apr. 19, 2007

(54) Surgical scalpel

(76) Inventors: David H. Masury, Kittery Point, ME (US); Edward P. Thompson, Portsmouth, NH (US)

Correspondence Address: Kevin M. Farrell;Pierce Atwood, Suite 350, One New Hampshire Avenue, Portsmouth, NH, 03801, US

(21) Appl. No.: 11/252,927

(22) Filed: Oct. 18, 2005

(51) Int. Cl. A61B 17/32

(52) U.S. Cl. 606/167

(57) ABSTRACT

The present invention includes a single-use surgical scalpel offering a variety of advantages relative to prior art devices. In preferred embodiments, the surgical scalpel of the present invention consists of three elements: a housing, a blade carrier, and a blade. The scalpel further includes an integrated light emitter selectively coupled to a power source when the scalpel is in use. The scalpel of the present invention further includes means for indicating that the blade has been previously advanced, including at least a viewing window disposed in the housing and a single use indicator integrated with the blade carrier.

### BACKGROUND OF THE INVENTION

1. Field of the Invention

The present invention relates generally to the field of medical devices, and more specifically to the field of instruments and devices for surgical procedures, especially including invasive procedures.

2. History of the Related Art

Surgical scalpels are used by medical personnel in connection with surgical procedures ranging from simple outpatient procedures to extraordinarily complex procedures carried out under general anesthesia. When working with such a sharp instrument, there is always a danger to medical personnel of inadvertent puncture of the skin. Aside from the injury itself, the more important concern for the punctured individual relates to the potential spread of disease from a non-sterile instrument.

The use of surgical scalpels having a retractable blade has been suggested as a means for reducing the number of such puncture wounds. Indeed, a number of patents have been issued which are directed toward surgical scalpels having retractable blades. These include, for example, U.S. Pat. Nos. 5,531,754, 6,254,621, 6,041,505, 6,022,364, 6,015,419, 5,908,432, and 5,779,724. Absent from the disclosure of these referenced U.S. patents, and any other publication of which Applicant is aware, is a means for illuminating the area in front of the blade that is contingent upon the blade being in a forward or exposed position. Those surgical scalpels that have incorporated light sources therein have not done so in such a manner that the exposure of the blade is inexorably tied to the operation of the light source. As such, there is a need in the art for a surgical scalpel that integrates a retractable blade and illuminating means in a functional sense.

### SUMMARY OF THE PRESENT INVENTION

The present invention relates to a single-patient surgical scalpel offering a variety of advantages relative to prior art devices. In preferred embodiments, the surgical scalpel of the present invention consists of three elements: a housing, a blade carrier, and a blade. The scalpel housing is preferably molded from plastic in an injection molding procedure. As will be seen by reference to the attached drawings, the housing consists of two halves that are either ultrasonically welded together, or held closed by means of snap together features in the assembled device with the blade carrier and blade in place. While the two halves can be molded independently, in preferred embodiments the two halves are molded as a single unit with the two halves being joined by a live hinge.

When assembled, with the two halves ultrasonically welded together, the molded plastic housing has an upper and a lower radiused edge, and interior and exterior wall surfaces. In preferred embodiments, the interior walls of the molded plastic housing are ribbed, with the innermost surfaces of the ribs defining a channel for the slidable engagement of a blade carrier. The use of molded ribs, rather than simply employing an unribbed inner wall surface, offers well-known advantages in the injection molding process. In addition, the ribbed bearing surface provides decreased friction in blade carrier movement when compared to the frictional forces encountered with a non-ribbed bearing surface. Other features of the channel include, an open end through which a surgical blade is extended during use, a slot through the lower radiused edge for slidable engagement of an actuator/locking fin, a single-use indicator window, and a retaining element for engaging a distal end of a serpentine spring.

The molded plastic blade carrier, which is slidably engaged by the channel defined by the ribbed interior wall surface of the housing, includes a first end adapted for engaging the surgical blade, and a second end comprising a serpentine spring to assist in blade retraction, the serpentine spring having a proximal and a distal end. The blade carrier also includes a central portion that includes the actuator/locking fin and a single-use indicator.

The present invention further includes an integrated light emitter and power source that are selectively coupled only during use. If the blade is moved into a forward or exposed position, then the light emitter will be connected to the battery thereby illuminating the space in front of the blade. If a user attempts to retract the blade into the housing, then the connection between the battery and the light emitter is ceased, thereby preserving battery power during a surgical procedure.

These and other features and advantages of the present invention are described more fully below with reference to the following drawings.

### BRIEF DESCRIPTION OF THE DRAWINGS

FIG. 1 is a perspective view of the surgical scalpel of the present invention according to one preferred embodiment.

FIG. 2 is a plan view of the housing portion of the surgical scalpel of the present invention, in an unassembled form.

FIG. 3 is a front, end view of the housing of the device of the present invention, in assembled form.

FIG. 4 is a bottom perspective view of the surgical scalpel of the present invention showing two-mated halves with blade carrier and blade in extended position.

FIG. 5 is a left-side plan view of the blade carrier of the present invention with the scalpel blade omitted.

FIG. 6 is a top plan view of the blade carrier of the present invention.

FIG. 7 is a magnified portion of the blade carrier in accordance with the present invention as shown in FIG. 5.

### DESCRIPTION OF PREFERRED EMBODIMENTS

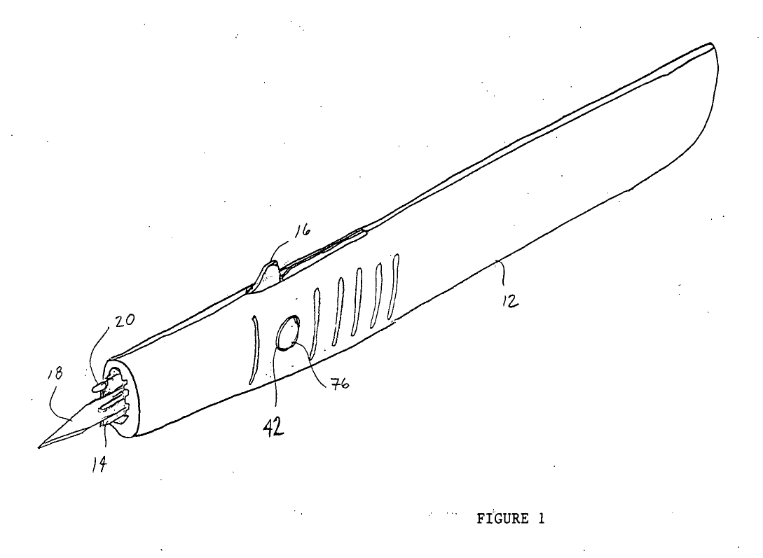

The present invention includes a surgical scalpel 10 having a single-patient retractable surgical blade 18 and a lighting source 20 that is activated in response to extension or use of the blade 18. As shown in FIG. 1, the scalpel 10 includes a molded plastic housing 12 that encompasses a blade carrier 14. The blade carrier 14 is adapted for carrying the blade 18 and is controlled via a fin or actuator 16. The lighting source 20 is integral with the blade carrier 14 and preferably disposed near the cutting edge of the blade 18; and the lighting source 20 is adapted to be activated only in response to a user moving the blade 18 into an extended, exposed position, as discussed in detail below.

The scalpel 10 further includes means for determining whether the blade 18 has been advanced, including for example a single-use indicator window 42 disposed in the housing 12. The window 42 is adapted for viewing a single use indicator 76 that is integrated with the blade carrier 14 such that when the blade carrier 14 is retracted within the housing 12, the single use indicator 76 will be disposed within the window 42. In one preferred embodiment, discussed below, the single use indicator 76 is a convex bubble that will be distorted when the blade 18 is advanced. Subsequently, if the blade 18 is retracted, then the single use indicator will be disposed within the window 42 such that a user can readily determine that the scalpel 10 has been previously used. In alternative embodiments, the single use indicator 76 may be of another geometry suitable for indicating that the blade 18 has been advanced while permitting later advancement of the blade 18 during a protracted surgical procedure.

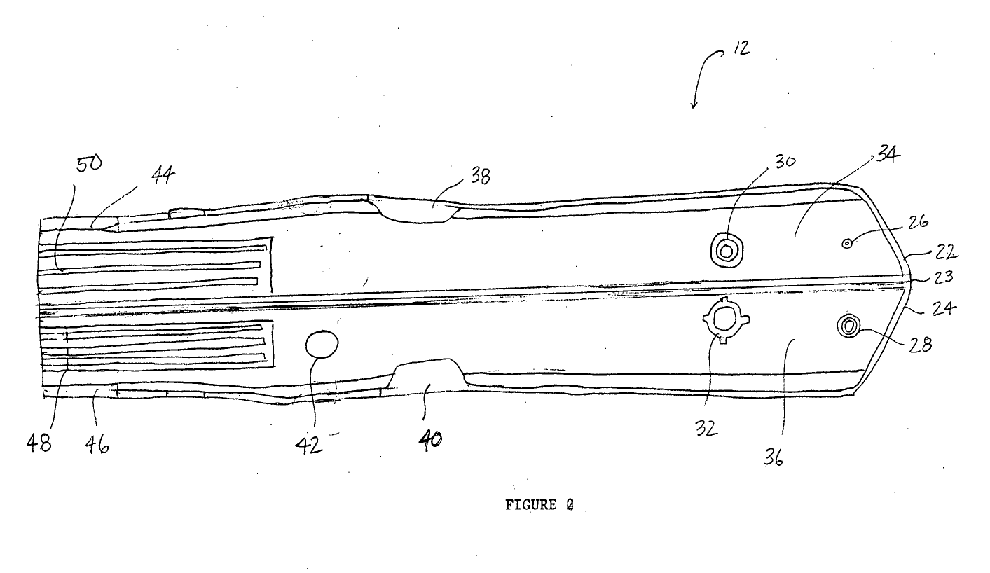

FIG. 2 depicts the housing 12 of the present invention. The housing 12 consists of a first half 22 and a second half 24 which are mated in the assembled device. While the first half 22 and the second half 24 can be independently molded prior to assembly, in preferred embodiments, the two halves are molded as a single unit and joined by a live hinge 23 which runs longitudinally and defines a common edge of each half. The live hinge 23 is essentially a thinly molded plastic strip which holds the two halves together and, by virtue of its thinness, enables the two halves to mate without breaking the hinge. Also shown in FIG. 1 is a molded press fit with male 26 and female 28 elements designed to hold the mated halves 22, 24 together in the assembled device prior to ultrasonic welding. Ultrasonic welding flanges 38, 40, 44, 46 are provided to effectively and permanently weld the halves 22, 24 together following assembly. Alternatively, permanent type snap together features (not illustrated) may be used in place of the ultrasonic welding to assemble the halves 22, 24.

The interior walls 34, 36 of the respective halves 22, 24 define a plurality of molded ribs 50 that define a channel for slidable engagement of a blade carrier. The use of a ribbed bearing surface, as opposed to unribbed interior wall surfaces, offers advantages in the injection molding process. The ribs 50 create a thicker and stiffer wall section whole reducing the volume of the material (relative to a solid wall of equal thickness). The reduction in thick sections prevents sink marks from occurring on the outer surfaces of the handle. These sink marks are caused by the shrinkage of the plastic during the cooling portion of the molding cycle. In addition, ribbed bearing surfaces offer less frictional resistance to a moving blade carrier than would unribbed interior wall surfaces.

A molded retaining element 30 is provided to anchor the distal end of a serpentine spring element of the blade carrier. The retaining element 30 is dimensioned for a secure press fit into a molded receiving element 32. In preferred embodiments, the retaining element 30 is an anchoring boss that engages a ring on the distal end of the serpentine spring, as described more fully below. The window 42 is provided to ensure that the scalpel 10 of the present invention is at least a single-patient device, as described further herein.

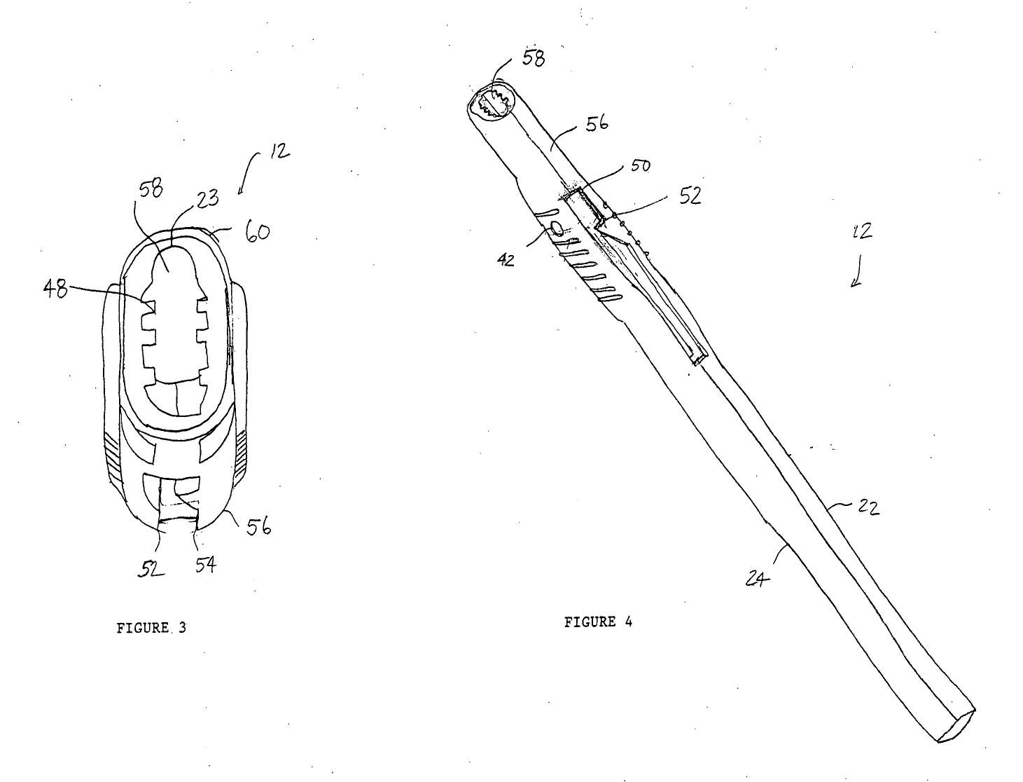

FIG. 3 is an end view of the housing 12 of the present invention, illustrating a proximal end of the assembled housing 12, which contains an opening 58 in communication with the interior channel of the housing 12. Also visible in this view is a shimming end 48 of a rib 50, a feature that is discussed in more detail below. A top edge 60 and a bottom edge 56 of the housing 12 are radiused for comfort and control.

The housing 12 of the present invention is shown assembled in perspective from below in FIG. 4, omitting the blade carrier 14 and associated elements. The lower edge of the assembled housing 12 contains a slot 50 for slidable engagement of the actuator 16, which is an element of the blade carrier 14 shown in FIG. 1. In preferred embodiments, a molded ramp 52 extends from the first half 22 of the housing 12 and cooperates with the second half 24 of the housing to define the slot 50. The molded ramp 52 is preferably angled in such a manner so as to retain the blade carrier in a blade-extended position during use.

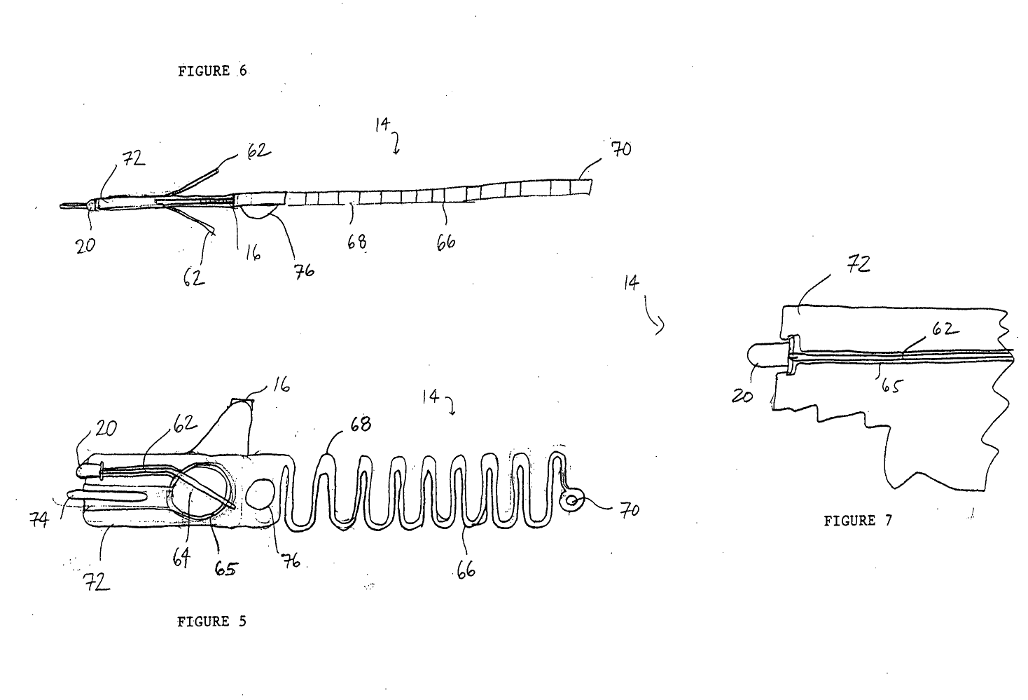

Referring to FIG. 5, the blade carrier 14 of the present invention is shown in a left-side plan view. The blade carrier 14 generally consists of three portions: a first end comprising a surgical blade engagement mold 74 adapted for receiving and securing a surgical blade, a second end comprising a serpentine spring 66 to assist in blade retraction, the serpentine spring having a proximal 68 and a distal end 70, and a central portion 72 including the actuator 16 and a recess or cavity 65 within which a battery 64 is disposable. The central portion 72 further includes the single use indicator 76, represented again as a bubble or protrusion that is adapted for viewing within the window 42 of the housing 12 when the blade 18 is in a retracted position. Alternative geometries of the single use indicator 76, such as those that permit repeated advancement and retraction of the blade 18 are also applicable to the present invention.

One of the many advantages of the present invention is that the blade carrier can be molded as a single unit from plastic. The use of a serpentine spring, rather than a metal coil spring, enables this advance over the art. Also shown is a molded anchoring ring 70 defining the distal end of the serpentine spring 66, the anchoring ring 70 adapted for engaging the molded retaining element 30 shown in FIG. 2.

The blade carrier 14 of the present invention further includes a lighting system including a light emitter 20 that is electrically connected to an electrical lead 62, depicted in FIG. 5 as a wire lead extending between the light emitter 20 and the battery 64. The light emitter 20 is preferably one of a filament-type bulb or a light emitting diode, both of which are adapted for use in conjunction with a low voltage battery 64 as contemplated herein. Other types of low-voltage lighting elements are equally applicable to the present invention, although the aforementioned options are preferred for their low power consumption and high intensity. The battery 64 is preferably disk-shaped and therefore disposable within the cavity 65 defined by the central portion 72 of the blade carrier 14.

FIG. 6 is a top plan view of the blade carrier 14 of the present invention. Again, the single use indicator 76 is shown as a bubble or protrusion from the central portion 72 of the blade carrier 14, although alternative geometries are easily contemplated as described herein. As shown, the electrical lead 62 includes a pair of lead wires that are connectable to the battery 64 should electrical contact be made. The electrical lead 62 wires are connected on one end to the light emitter 20, and pass through a portion of the cavity 65 that is defined within the blade carrier 14. The electrical lead 62 wires remain unconnected on the other end. Preferably, the electrical lead 62 wires are angled away from the central portion 72 in such a manner that they are incapable of making contact with the battery 64 without being forced to do so. In such a manner, the light emitter 20 is only selectively operational.

The central portion 72 of the blade carrier 14 and the electrical lead 62 wires are configured such that the latter will only make contact with the battery 64 when the blade carrier 14 is moved into its extended position. Referring back to FIG. 2, the ribs 50 of the housing 12 function to both secure the blade carrier 14 in its forward position and furthermore to act as a lever to push the electrical lead 62 wires into electrical contact with the battery 64 in the forward position. Accordingly, when the scalpel 10 of the present invention is not in use, the light emitter 20 will not be operable. If a user moves the actuator 16 and the blade carrier 14 into the forward position, the ribs 50 along the interior of the housing 12 will provide the necessary mechanical pressure to force the electrical connection between the battery 64 and the electrical lead 62, thus providing power to the light emitter 20. As such, whenever the scalpel 10 of the present invention is in use, the light emitter 20 will be operational. If the scalpel 10 is not in use, or if the blade 18 is otherwise retracted, then the light emitter 20 will not be operational.

FIG. 7 is a magnified view of the portion of the blade carrier 14. As shown, the light emitter 20 is arranged in the blade carrier 14 such that it will illuminate the space near the blade 18 (not visible in FIG. 7). In a preferred embodiment, the present invention is single-use, and most preferably the single use indicator 76 will only be functional for one use, thus ensuring that the scalpel 10 of the present invention is never reused. In one embodiment, as the blade carrier 14 is moved into the forward position, the single use indicator 76 is visibly distorted by the interior of the housing 12 such that subsequent retraction of the blade carrier 14 moves the single use indicator 76 into the window, wherein a user can easily identify that the blade 18 has been previously advanced.

However, in alternate embodiments, the present invention is adapted for use as a single-patient device in which the blade 18 can be repeatedly retracted and advanced during a prolonged procedure. In this instance, it is not entirely necessary for the single use indicator 76 to be shaped in a manner indicative of a single use, i.e. as a convex bubble or protrusion. Rather, the single use indicator 76 can be disk shaped or otherwise relatively unobtrusive so as to permit repeated use of the scalpel 10 on a single patient. Alternatively, for those applications or surgical procedures in which the blade 18 may be repeatedly advance and retracted, the present invention can be readily configured without the single use indicator 76 and the window 42.

In a single-mold embodiment of the present invention, not shown, the blade carrier 14 is integrally molded with the housing 12 and a second live hinge connects the two components. While functionally similar, the single-mold embodiment of the present invention may further decrease the risk of reuse of the device because the blade carrier 14 cannot be removed from the housing 12.

As described herein, the present invention includes a surgical scalpel that includes an integrated light emitter and power source. The light emitter is positioned such that it emits light during use, and further such that any retraction of the blade severs electrical connection between the light emitter and the power source. While described with reference to its particular preferred embodiments, the present invention also encompasses those various modifications that can be readily made by those skilled in the art that fall within the scope of the following claims.

We claim:

1. A surgical scalpel, comprising:
a molded plastic housing defining a channel for slidable engagement of a blade carrier adapted for securing a surgical blade;
a molded plastic blade carrier comprising a first end adapted for engaging the surgical blade at second end comprising a serpentine spring to assist in blade retraction, the serpentine spring having a proximal and a distal end and a central portion including an actuator; and
a lighting system integrated into the molded plastic blade carrier, the lighting system including a light emitter disposed at the first end of the blade carrier, a battery disposed at the central portion of the blade carrier, and an electrical lead selectively connecting the light emitter and the battery.

2. The scalpel of claim 1 further wherein the molded plastic housing comprises an upper and a lower radiused edge, and ribbed interior wall surfaces, the ribbed interior wall surfaces of the molded plastic housing defining the channel through which the blade carrier is slidable.

3. The scalpel of claim 1 wherein the electrical lead is adapted to conduct current between the battery and the light emitter in response to the blade carrier is in an extended position.

4. The scalpel of claim 1 wherein the light emitter is a filament bulb.

5. The scalpel of claim 1 wherein the light emitter is a light emitting diode.

6. The scalpel of claim 1 wherein the central portion of the blade carrier defines a cavity within which the battery is disposable.

7. The scalpel of claim 1 further comprising a ribbed interior wall disposed within the molded plastic housing further defining the channel through which the blade carrier is slidable.

8. The scalpel of claim 1 wherein the single-use indicator on the molded plastic blade carrier comprises a flexible plastic convex element which is positioned in, and viewable through, the single-use indicator window in the molded-plastic housing when the blade is in the retracted position, the convex plastic element being irreversibly altered when the blade carrier is advanced to expose the surgical blade.

9. The scalpel claim 1 wherein the exterior walls of the molded plastic housing further comprise raised molded fins in critical gripping locations.

10. The scalpel of claim 1 wherein the molded plastic housing is a unitary construct, having a live hinge that defines the central line of the longitudinal axis in a hinge-open view.

11. The scalpel of claim 1 wherein the retaining element for engaging a distal end of a serpentine spring comprises a molded boss that engages a ring formed on the distal end of the serpentine spring.

12. The scalpel of claim 1 wherein the electrical lead is selectively couplable to the battery such that retraction of the blade carrier severs the electrical lead between the battery and the lighting system.
